(19) United States
(12) Patent Application Publication
(10) Pub. No.: US 2004/0047884 A1
(43) Pub. Date: Mar. 11, 2004

(54) Long-lasting powder cosmetic composition comprising a specific adhesive material

(76) Inventors: Pascale Bernard, Gabriel Peri, Sucy-En-Brie (FR); Anke Hadasch, Saint Marcel, Paris (FR); Beatrice Toumi, Verrieres-Le-Buisson (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/312,805

(22) PCT Filed: Jul. 9, 2001
(86) PCT No.: PCT/FR01/02207

(30) Foreign Application Priority Data
Jul. 13, 2000 (FR) 00/09215

(51) Int. Cl. A61K 7/00

(52) U.S. Cl. 424/401

(57) ABSTRACT

The invention concerns a powder composition, in particular cosmetic and/or physiological, for skin make-up or care, capable of being in free or compact form, comprising at least a powder compound and a binder containing at least a specific adhesive material. Said composition exhibits remarkable non-transfer and long-lasting properties. The invention also concerns the use of an adhesive material in such a composition.

The present invention relates to a pulverulent composition comprising an adhesive material intended in particular for the fields of cosmetics and hygiene. More especially, the invention relates to a long-lasting and transfer-resistant pulverulent makeup and/or care composition for both human facial skin and body skin.

This composition may optionally be in the form of a free or compact makeup powder for the face and more especially for the cheeks and the eyelids. It may especially be in the form of a rouge, an eye shadow or a blusher, or alternatively in the form of a face powder, body powder or deodorant powder. It may be compacted, pressed or cast, in the form of a stick, a dish or any other form.

Face powders are often intended to make the skin matt and/or to color the skin. They generally contain a large amount of pulverulent compounds formed of pigments and/or fillers, bound together with the aid of a phase known as a binder, this phase generally being fatty and comprising an oil or a mixture of oils that are liquid at room temperature. This fatty phase may also comprise waxes and optionally additives, for instance cosmetic or dermatological active agents. The powders may also contain surfactants.

When the powders are applied to skin or the lips, they have the drawback of transferring, i.e. of coming off at least partially, leaving marks on certain supports with which they may come into contact, and especially an item of clothing or the skin or alternatively a glass, a cup or a cigarette. This transfer occurs especially during rubbing. This results in mediocre persistence of the applied product, making it necessary for the application of the powder or makeup to be freshened regularly. Moreover, the appearance of these unacceptable marks, especially on blouse collars, may put certain women off using this type of makeup.

Furthermore, these powders have a tendency to disappear from the human face or body, due to the production of sweat and sebum by the skin. Thus, the powder is often found to migrate in the folds, wrinkles and fine lines of the skin, causing lines to appear; this is especially the case in the wrinkles around the eyes and the folds of the eyelids. In the case of face powders or blushers, the migration of the powder is reflected by marking of certain deep wrinkles, highlighting the user's age, and also a disappearance of the color. The makeup then becomes unsightly and heterogeneous. An increase in the sheen of the makeup in the course of the day may also be observed, making it necessary to make another application or spot touching-up of the makeup.

To absorb sweat and sebum, porous fillers and optionally fluorinated binders are generally used, which “repel” the sebum and sweat, or alternatively fillers and pigments coated with fluorinated agents. This type of powder has the drawback especially of being expensive, thus not allowing marketing to the general public. In addition, these fluorinated compounds often have problems of harmfulness and toxicity.

To improve the adhesion of the powder on the skin, use may also be made of binders comprising compounds that have a certain sticky nature such as, for example, petroleum jelly. However, the composition is very often too cohesive, too sticky and is very difficult to crumble: the user cannot easily take up the portion of powder that she desires to use with the fingers or a powder puff.

However, it is fundamental, in particular for a makeup product, that the product should be able to be taken up in an ideal manner, i.e. that not only should the adequate amount of product (not too much so as not to lose product needlessly, but sufficient to produce a makeup effect) be able to be taken up simply, using the fingers or a sponge or even directly onto the skin of the body, for example, but also that the integrity of the product should be conserved while it is being taken up: the product should not break, for example via a shear phenomenon, but it should not fray or be sticky either.

There is thus still a need for a pulverulent composition that does not have the above drawbacks, and that especially has “transfer-resistance” properties and is long-lasting, while remaining easy to crumble, this composition allowing a uniform makeup result.

The Applicant has found, entirely surprisingly, that the use of a particular adhesive material in a pulverulent cosmetic or physiological composition produces a makeup of very good staying power, which does not transfer at all and is water-resistant, the composition moreover being easy to break down.

A subject of the present invention is thus a pulverulent cosmetic and/or physiological composition comprising at least one binder and at least one pulverulent compound, characterized in that the binder comprises at least one adhesive material satisfying the following conditions:

G′ (2 Hz, 35° C.)≧10³ Pa, and

G′ (35° C.)≦10⁸ Pa, preferably G′ (35° C.)≦10⁷ Pa,

G′ (2×10⁻² Hz, 35° C.)≦3×10⁵ Pa, and

G″/G′ (0.2 Hz, 35° C.)≧0.35

in which:

G′ (2 Hz, 35° C.) is the elastic shear modulus of said adhesive material, measured at a frequency of 2 Hz and at a temperature of 35° C.,

G′ (2×10⁻² Hz, 35° C.) is the elastic shear modulus of said adhesive material, measured at a frequency of 2×10⁻² Hz and at a temperature of 35° C.,

G′ (35° C.) is the elastic shear modulus of said adhesive material, measured at a temperature of 35° C. for any frequency between 2×10⁻² and 2 Hz,

G″(0.2 Hz, 35° C.) is the viscous shear modulus of said adhesive material, measured at a frequency of 0.2 Hz and at a temperature of 35° C.,

G′ (0.2 Hz, 35° C.) is the elastic-shear modulus of said adhesive material, measured at a frequency of 0.2 Hz and at a temperature of 35° C.

The condition “10⁸ Pa≧G′ (35° C.), preferably 10⁷ Pa≧G′ (35° C.)” does not mean that G′ (35° C.) is constant throughout this frequency range of between 2×10⁻² and 2 Hz, but that, irrespective of the value of the frequency over this range, the condition is satisfied.

EP 0 850 649 describes adhesive articles for treating the skin. However, said document does not describe the use of adhesive materials according to the present invention in makeup powders for the face or for the body.

The composition according to the invention has excellent staying power properties on the skin and excellent “transfer-resistance” properties. It adheres well to the skin. It is resistant to friction, sweat, sebum and water. It breaks down easily. It allows a homogeneous deposition over time on the skin and a uniform makeup. Once applied to the skin, it does not migrate and has very good color fastness over time. The composition spreads easily and is very comfortable to wear. It is pleasant to apply and to wear throughout the day. It is not greasy and does not shine over time. It has a pleasant, creamy feel.

Another subject of the invention is the use, in or for the manufacture of a pulverulent cosmetic or physiological composition, of at least one adhesive material as defined above to reduce, or even eliminate, the transfer and/or migration, and/or to increase the staying power, of the composition applied to human skin onto a support placed in contact with the deposit.

Another subject of the invention is the use, in or for the manufacture of a pulverulent cosmetic or physiological composition, of at least one adhesive material as defined above to increase the color fastness of the composition over time.

A subject of the invention is also a nontherapeutic care and/or makeup process for human skin, which consists in applying to the skin a cosmetic composition as defined above.

The composition according to the invention comprises a binder comprising at least one particular adhesive material. For the purposes of the present invention, the term “binder” means a phase that is liquid or semisolid at room temperature (25° C.), preferably having a viscosity ranging from 10⁻³ Pa.s to 10⁵ Pa.s, this viscosity being measured during tests of flow in torsion/shear on a rheometer of “Haake RS50” type at a temperature of 25° C. for a shear rate of 2 s⁻¹, for example in cone-plate geometry. The binder is a nonpulverulent phase intended to bind the solid particles of the pulverulent phase and to ensure their homogeneous dispersion and their cohesion within the final composition. For the purposes of the present invention, the term “material” means a polymer or a polymer system that may comprise one or more polymers of different nature. This adhesive material must have a certain level of bonding power defined by its viscoelastic properties.

The viscoelastic properties of a material are conventionally defined by two characteristic values, which are as follows:

the elastic shear modulus, which represents the elastic behavior of the material for a given frequency and which is conventionally written as G′,

the viscous shear modulus, which represents the viscous behavior of the material for a given frequency, and which is conventionally written as G″.

These magnitudes are especially defined in the “Handbook of Pressure Sensitive Adhesive Technology” 3rd Edition, D. Satas, Chapter 9, pp. 155 to 157.

The adhesive materials that may be used according to the present-invention have viscoelastic properties that are measured at a reference temperature of 35° C. and within a certain frequency range. The elastic modulus of the material is measured in particular at three different frequencies:

at low frequency, i.e. at 2×10⁻² Hz,

at an intermediate frequency, i.e. at 0.2 Hz,

at high frequency, i.e. at 2 Hz,

and the viscous modulus at a frequency of 0.2 Hz.

These measurements allow the change in the bonding power of the adhesive material over time to be evaluated.

The adhesive materials which can be used according to the present invention satisfy the following four conditions:

G′ (2 Hz, 35° C.)≧10³ Pa, and

G′ (35° C.)≦10⁸ Pa, preferably G′ (35° C.)≦10⁵ Pa,

G′ (2×10⁻² Hz, 35° C.)≦3×10⁵ Pa, and

G″/G′ (0.2 Hz, 35° C.)≧0.35

in which:

G′ (2 Hz, 35° C.) is the elastic shear modulus of said adhesive material, measured at a frequency of 2 Hz and at a temperature of 35° C.,

G′ (2×10⁻² Hz, 35° C.) is the elastic shear modulus of said adhesive material, measured at a frequency of 2×10⁻² Hz and at a temperature of 35° C.,

G′ (35° C.) is the elastic shear modulus of said adhesive material, measured at a temperature of 35° C. for any frequency between 2×10⁻² and 2 Hz,

G′ (0.2 Hz, 35° C.) is the viscous shear modulus of said adhesive material, measured at a frequency of 0.2 Hz and at a temperature of 35° C.,

G′ (0.2 Hz, 35° C.) is the elastic shear modulus of said adhesive material, measured at a frequency of 0.2 Hz and at a temperature of 35° C.

These viscoelastic properties are measured during dynamic tests under sinusoidal stresses of low amplitude (small deformations, for example of 1%) performed at 35° C. over a frequency range of from 2×10⁻² to 20 Hz on a “Haake RS50” rheometer under a torsional/shear stress, for example in cone-plate geometry (for example with a cone angle of 10).

In one preferred form of the invention, the following applies:

G′ (2 Hz, 35° C.)≧10⁴ Pa.

In another preferred form of the invention, the following applies:

G′ (2×10⁻² Hz, 35° C.)≦5×10⁴ Pa.

More preferably, the adhesive materials according to the invention satisfy the following four conditions:

G′ (2 Hz, 35° C.)≧10⁴ Pa, and

G′ (35° C.)≦10⁸ Pa, preferably G′ (35° C.)≦10⁷ Pa,

G′ (2×10⁻² Hz, 35° C.)≦5×10⁴ Pa, and

G″/G′ (0.2 Hz, 35° C.)≧0.35.

The adhesive materials according to the invention may be chosen from adhesives of “pressure-sensitive adhesive” type for example, such as those mentioned in the “Handbook of Pressure Sensitive Adhesive Technology” 3rd Edition, D. Satas.

The proadhesive materials according to the invention are preferably adhesive polymers chosen from block and random copolymers comprising at least one monomer or a combination of monomers whose resultant polymer has a glass transition temperature of less than room temperature (25° C.), these monomers or monomer combinations possibly being chosen from butadiene, ethylene, propylene, isoprene, isobutylene and a silicone, and blends thereof. Examples of such materials are block polymers of the styrene-butadiene-styrene, styrene-(ethylene-butylene)-styrene or styrene-isoprene-styrene type, for instance those sold under the trade name “Kraton” from Shell Chemical Co. or “Vector” from Exxon.

The proadhesive materials according to the invention are preferably adhesive polymers chosen from:

polyurethanes,

acrylic polymers,

silicones,

butyl rubbers, especially from polyisobutylenes,

ethylene/vinyl acetate polymers,

polyamides optionally modified with fatty chains,

natural rubbers,

and blends thereof.

In one particularly advantageous form of the invention, the adhesive materials are chosen from polyisobutylenes with a relative molar mass Mv of greater than or equal to 10,000 and less than or equal to 150,000. More preferably, this relative molar mass is greater than or equal to 18,000 and less than or equal to 150,000.

This relative molar mass Mv may be evaluated by its viscosimetric mean, calculated according to the formula J₀=3.06×10⁻² Mv^{0.65}, in which J₀ is the Staudinger index (in cm³/g). This index is calculated from the flow time of a solution with a polyisobutylene concentration of C=0.01 g/cm³ in isooctane, through a capillary I of an Ubbelhode viscometer at 20° C. according to ISO standard 1628.

The relative molar mass Mv may also be evaluated by GPC (gel permeation chromatography) according to the following protocol: 200 μl of a 0.5% solution of polymer (adhesive material) are injected by means of a Waters 6000 A pump, the eluent being a 100% THF solution, flow rate 1 ml/min, at room temperature, through a set of 8 columns: μstyragel 500 Å+10⁴ Å+2×10³ Å+styragel HRO.5+2×HR1+HR5E (300×7.8 mm). The detection is performed on a Waters 410 refractometer and on a Waters 490 UV detector at a wavelength of 254 nm.

As commercial products that are particularly suitable for the present invention, mention may be made of polyisobutylenes with respective relative molar masses (viscosimetric average) of 40,000, 55,000 and 85,000 sold under the respective trade names “Oppanol B 10”, “Oppanol B 12” and “Oppanol B 15” by the company BASF, and blends thereof.

In another embodiment of the invention, the adhesive material according to the invention may also be a mixture of one or more polyisobutylenes with a relative molar mass of less than or equal to 10,000 and of one or more polyisobutylenes with a relative molar mass of greater than 150,000.

Among the polyisobutylenes with a relative molar mass of less than or equal to 10,000, mention may be made of the polyisobutylenes of molar mass 455 to 2,000 sold under the trade name “Napvis” by the company BP Chemicals or the polyisobutylenes sold under the name “Parleam” by the Ets B. Rossow et Cie.

Among the polyisobutylenes with a relative molar mass of greater than 150,000, mention may be made of the polyisobutylenes with respective relative molar masses of 200,000, 400,000 and 1,110,000 sold under the respective trade names “Oppanol B 30 SF”, “Oppanol B 50 SF” and “Oppanol B 100” by the company BASF, the polyisobutylenes with relative molar masses of between 900,000 and 2,200,000 sold under the trade name “Vistanex MM” by the company Exxon, and blends thereof.

The adhesive materials are preferably present in the composition according to the invention in a content ranging from 0.1% to 99%, preferably from 0.1% to 30% and more preferably from 0.1% to 10% by weight, relative to the total weight of the composition.

The binder may comprise, in addition to the adhesive material above, any cosmetically acceptable water-soluble, water-dispersible, liposoluble or lipodispersible compound conventionally used in cosmetics. Thus, the binder may be in the form of an anhydrous fatty phase (gel or solution), in the form of an aqueous phase (gel or solution), in the form of a dispersion, in the form of an aqueous dispersion of particles such as a latex or a pseudolatex, in the form of a nonaqueous dispersion of particles, in the form of an O/W or W/O emulsion, or of a multiple emulsion, optionally stabilized with one or more organized systems.

For the purposes of the present invention, the expression “organized systems” means inverse micelles or “lyotropic liquid crystal” structures which are formed at room temperature by mixing together several surfactants or mixing together surfactants and polar solvents or mixing together several polar solvents, the polar solvents being chosen, for example, from water, glycerol, panthenol, propylene glycol and butylene glycol, and/or mixtures thereof. The liquid crystal state is a state that is intermediate between the solid state and the liquid state. It is often referred to as the mesomorphic state. These organized systems are thermodynamically stable.

Depending on its nature, the adhesive material according to the invention may be present in the binder in dissolved form or in dispersed form, in an aqueous phase or in an anhydrous phase. The adhesive material according to the invention may thus be in the form of an aqueous dispersion of particles or in the form of an oily dispersion of particles.

When the binder according to the invention comprises a fatty phase, it can preferably comprise at least one cosmetically or physiologically acceptable oil chosen especially from carbon-based, hydrocarbon-based and/or silicone-based oils of mineral, animal, plant or synthetic origin, and mixtures thereof, provided that they are compatible with the intended use.

Mention may be made of hydrocarbon-based oils such as liquid paraffin or liquid petroleum jelly, mink oil, turtle oil, soybean oil, perhydrosqualene, sweet almond oil, beauty-leaf oil, palm oil, grapeseed oil, sesame seed oil, corn oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl alcohol or linolenyl alcohol, isostearyl alcohol or octyldodecanol; silicone oils such as polydimethylsiloxanes (PDMS), which are optionally phenylated such as phenyltrimethicones, or optionally substituted with aliphatic and/or aromatic groups, or with functional groups such as hydroxyl, thiol and/or amine groups; polysiloxanes modified with fatty acids, with fatty alcohols or with polyoxyalkylenes, and mixtures thereof.

Advantageously, at least one oil that is volatile at room temperature may be used. The term “volatile oil” means an oil that is capable of evaporating from the skin at room temperature in less than one hour. Preferably, the volatile oil has a viscosity ranging from 0.5 to 25 centistokes at 25° C. After evaporation of these oils, a nonsticky particle deposit is obtained on the skin or mucous membranes. Preferably, oils whose flash point is high enough to allow these oils to be used in the formulation are used. These volatile oils also facilitate the application of the composition to the skin.

These oils may be hydrocarbon-based oils or silicone oils optionally comprising alkyl or alkoxy groups at the end of a silicone chain or pendent.

As volatile silicone oils that may be used in the invention, mention may be made of linear or cyclic silicones containing from 2 to 7 silicon atoms, these silicones optionally comprising alkyl or alkoxy groups containing from 1 to 10 carbon atoms. Mention may thus be made especially of octamethylcyclotetrasiloxane, decamethylcyclopentasiloxane, hexadecamethylcyclohexasiloxane, heptamethylhexyltrisiloxane and heptamethyloctyltrisiloxane, and/or mixtures thereof.

Volatile hydrocarbon-based oils that may be mentioned include C₈-C₁₆ isoparaffins such as isooctane, isododecane, isodecane, heptane and isohexadecane, and/or mixtures thereof.

These volatile oils may be present in the composition according to the invention in a content ranging from 0.1% to 99% by weight, preferably from 0.1% to 60% and more preferably from 0.1% to 40%, relative to the total weight of the composition. Preferably, the weight ratio of volatile oil to the adhesive material according to the invention ranges from 1 to 20, more preferably from 1 to 10 and better still from 2 to 4.

The fatty phase may also comprise at least one wax, at least one gum and/or at least one pasty fatty substance, of plant, animal, mineral or synthetic origin, or even silicone-based, and mixtures thereof.

Among the waxes that are solid at room temperature, which may be present in the composition according to the invention, mention may be made of hydrocarbon-based waxes such as beeswax, carnauba wax, candelilla wax, ouricurry wax, Japan wax, cork fiber wax or sugar cane wax, paraffin wax, lignite wax, microcrystalline waxes, lanolin wax, montan wax, ozokerites, polyethylene waxes, the waxes obtained by Fischer-Tropsch synthesis, hydrogenated oils, fatty esters and glycerides that are solid at 25° C. Silicone waxes may also be used, among which mention may be made of alkyl, alkoxy and/or esters of polymethylsiloxane. The waxes may be in the form of stable dispersions of colloidal wax particles as may be prepared according to known methods, such as those of “Microemulsions Theory and Practice”, L. M. Prince Ed., Academic Press (1977), pages 21-32. Mention may be made of jojoba oil as a wax that is liquid at room temperature.

The waxes may be present in a proportion of from 0.1% to 30% by weight relative to the total weight of the composition.

The pasty fatty compounds may be defined by means of at least one of the following physicochemical properties:

a viscosity from 0.1 to 40 Pa.s (1 to 400 poises) and preferably 0.5 to 25 Pa.s, measured at 40° C. with a Contraves TV rotary viscometer equipped with an MS-r3 or MS-r4 rotor at a frequency of 60 Hz,

a melting point of 25-70° C. and preferably 25-55° C.

The compositions of the invention may also comprise at least one alkyl, alkoxy or phenyl dimethicone such as, for example, the product sold under the name “Abil Wax 2440” by the company Goldschmidt.

The compositions according to the invention may also comprise at least one silicone resin comprising a combination of units R₃SiO_{1/2}, R₂SiO_{2/2}, RSiO_{3/2} and SiO_{4/2}, in which R denotes an alkyl radical containing from 1 to 6 carbon atoms.

The fatty phase may also contain at least one liposoluble dye. This liposoluble dye is, for example, Sudan red, DC Red 17, DC Green 6, β-carotene, soybean oil, Sudan brown, DC Yellow 11, DC Violet 2, DC Orange 5, or quinoline yellow, and mixtures thereof. It can represent from 0.01 to 20% of the total weight of the compositions, and better still from 0.1% to 6%.

The fatty phase can represent from 0.1% to 40%, preferably from 0.1% to 20%, by weight relative to the total weight of the final composition.

When the binder according to the invention comprises an aqueous phase, it may comprise water, a floral water such as cornflower water and/or a mineral water such as eau de Vittel, eau de Lucas or eau de La Roche Posay and/or a spring water.

The aqueous phase may also comprise solvents other than water, such as, for example, primary alcohols such as ethanol and isopropanol, glycols such as propylene glycol, butylene glycol, dipropylene glycol or diethylene glycol, glycol ethers such as mono-, di- or tripropylene glycol (C₁-C₄)alkyl ethers, and mono-, di- or triethylene glycol, and mixtures thereof.

The aqueous phase may also comprise at least one water-soluble colorant chosen from the colorants that are common in the field under consideration, such as the disodium salt of ponceau, the disodium salt of alizarin green, quinoline yellow, the trisodium salt of amaranth, the disodium salt of tartrazine, the monosodium salt of rhodamine, the disodium salt of fuchsin, or xanthophyll, and mixtures thereof.

The aqueous phase may also comprise any water-soluble or water-dispersible compound that is compatible with an aqueous phase, such as gelling agents, film-forming polymers, thickeners, surfactants, and mixtures thereof.

Preferably, the aqueous phase is present in the compositions of the invention in a content ranging from 0.1% to 40% by weight and preferably from 0.1% to 20% by weight, relative to the total weight of the composition.

The binder of the composition according to the invention may also comprise at least one O/W or W/O surfactant conventionally used in cosmetics.

The composition according to the invention additionally comprises at least one pulverulent compound. The pulverulent compounds may be chosen from the pigments and/or nacres and/or fillers and/or mixtures thereof usually used in cosmetic compositions.

Advantageously, the pulverulent compounds are present in a content ranging from 60% to 99.9% and more preferably from 80% to 99.9% by weight, relative to the total weight of the composition.

The pigments may be white or colored, and mineral and/or organic. Among the mineral pigments that may be mentioned are titanium dioxide, optionally surface-treated, zirconium oxide or cerium oxide, also iron oxide or chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminum.

The nacreous pigments may be chosen from white nacreous pigments such as mica coated with titanium or with bismuth oxychloride, colored nacreous pigments such as titanium mica with iron oxides, titanium mica with, especially, ferric blue or chromium oxide, titanium mica with an organic pigment of the abovementioned type, and also nacreous pigments based on bismuth oxychloride.

The fillers may be mineral or organic, and lamellar or spherical. Mention may be made of talc, mica, silica, kaolin, Nylon powders (Orgasol from Atochem or Nylon 12), poly-β-alanine powders and polyethylene powders, Teflon, bismuth oxychloride, lauroyllysine, starch, boron nitride, tetrafluoroethylene polymer powders, hollow microspheres such as Expancel (Nobel Industrie), Polytrap (Dow Corning) and silicone resin microbeads (for example Tospearls from Toshiba), precipitated calcium carbonate, magnesium carbonate, magnesium hydrocarbonate, hydroxyapatite, hollow silica microspheres (Silica Beads from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate.

The composition may also comprise any compound usually-used in such compositions, such as thickeners, antioxidants, fragrances, preserving agents or active agents.

As cosmetic, dermatological or hygiene active agents that may be used in the composition of the invention, mention may be made of moisturizers, vitamins, essential fatty acids, sphingolipids and sunscreens. These active agents are present in an amount generally used in powders. In particular, they are present in a proportion of from 0.001 to 20% relative to the total weight of the composition.

Needless to say, a person skilled in the art will take care to select this or these optional additional compound(s), and/or the amount thereof, such that the advantageous properties of the composition according to the invention are not, or are not substantially, adversely affected by the envisioned addition.

In one particular embodiment of the invention the compositions according to the invention may be prepared in the usual manner by those skilled in the art. They may be in the form of a compact powder, for example, in the form of a stick or dish that may be used by taking up with the finger or with a sponge: in particular, they find use as blushers, face powders, eye shadows, lip powders, care or makeup powders for the face or the body, or deodorant powders. They may also-be in the form of a free powder, especially as face powders, care or makeup powders for the face or the body, or deodorant powders.

These compositions for topical application may also constitute a cosmetic composition, dermatological composition, protective or hygiene composition, medicated composition or care composition for the face, for the neck, for the hands or for the body (for example fragranced or unfragranced anhydrous care powder, or antisun powder), a makeup composition (for example a makeup gel) or an artificial tanning composition.

The invention is illustrated in greater detail by the examples that follow.

### EXAMPLE 1

The Applicant prepared the composition in powder form below:

A) Pulverulent Phase:

| | |
|---|---|
| talc | 65.19% |
| iron oxides | 2.51% |
| Nylon powder | 10% |
| titanium oxide | 3% |
| methylsilsesquioxane resin | 5% |
| preserving agent | 0.3% |

B) Binder (Fatty Phase):

| | |
|---|---|
| polyisobutylene of molecular mass | 4% |
| 40,000 sold under the trade name | |
| “Oppanol B 10” by the company BASF | |
| isododecane | 10% |

The product “Oppanol B 10” has the following characteristics, measured according to the methods described in the present patent application:

G′ (2 Hz, 35° C.)=5×10⁴ Pa

G′(2×10⁻² Hz, 35° C.)=1×10² Pa

G″/G′ (0.2 Hz, 35° C.)=2.75

Procedure:

The binder (phase B) is prepared in the following manner: the polyisobutylene are is mixed with the isododecane with stirring at room temperature until a homogeneous liquid is obtained.

The compounds of phase A are then mixed together in a turbomixer. This mixture is then ground using a pin mill or an airjet mill. Finally, the binder (phase B) is added dropwise to phase A with stirring in a turbomixer whose temperature is kept constant at 20° C. The two phases are mixed together until a homogeneous product is obtained.

The composition obtained has excellent staying power over time. It does not transfer and no phenomenon of migration is observed.

### EXAMPLE 2

The Applicant prepared composition 2 below:

A) Pulverulent Phase:

| | |
|---|---|
| talc | 58.9% |
| iron oxides | 2.8% |
| Nylon powder | 10% |
| titanium oxide | 3% |
| methylsilsesquioxane resin | 5% |
| preserving agent | 0.3% |

B) Binder (Fatty Phase):

| | |
|---|---|
| polyisobutylene of molecular mass | 0.8% |
| 1,110,000 sold under the trade name | |
| “Oppanol B 100” by the company BASF | |
| polyisobutylene of molecular mass | 3.2% |
| 455 sold under the trade name | |
| “Napvis D 07” by the company | |
| BP Chemicals | |
| isododecane | 16% |

This composition was prepared according to the same method as in example 1, the binder being prepared by mixing the polyisobutylenes with the isododecane with stirring at room temperature until a homogeneous liquid was obtained.

The adhesive material according to the invention is thus the mixture of 0.8% of the polyisobutylene “Oppanol B100” and 3.2% of the polyisobutylene “Napvis D 07”: this adhesive material has the following characteristics, measured according to the methods described in the present patent application:

G′ (2 Hz, 35° C.)=7×10³ Pa

G′ (2×10⁻² Hz, 35° C.)=2×10³ Pa

G″/G′ (0.2 Hz, 35° C.)=0.35

A powder that has excellent staying power over time and on the skin is obtained.

### EXAMPLE 3 (COMPARATIVE)

The Applicant prepared composition 3 (comparative) below:

A) Pulverulent Phase:

| | |
|---|---|
| talc | 67.6% |
| yellow iron oxide | 1.9% |
| red iron oxide | 0.62% |
| black iron oxide | 0.63% |
| Nylon powder | 10.52% |
| titanium oxide | 3.15% |
| methylsilsesquioxane resin | 5.26% |
| methyl p-hydroxybenzoate | 0.32% |

B) Binder (Fatty Phase):

| | |
|---|---|
| isododecane | 10% |

This composition was prepared according to the same method as in example 1, the binder comprising no polyisobutylene.

The transfer-resistance properties of compositions 1 (example 1 according to the invention) and 3 (comparative example 3) were tested on 10 models in the following manner: each composition was applied comparatively to half the face and the neck. After applying the makeup, a “collar” made of fabric was placed around the neck. This fabric was removed 30 minutes later in order to visualize the amount of powder transferred onto the fabric.

In 70% of cases, the amount of powder transferred onto the fabric is markedly less for composition 1 (example 1). The models were asked to grade the transfer-resistance effect on a scale from 0 to 7, the value 0 being given for a composition that does not transfer at all, and the value 7 being given for a composition that transfers totally. Composition 1 according to the invention obtains an average of 3, as compared with an average of 4.2 for the comparative composition 3. This shows that the transfer-resistance properties of example 1 (containing the adhesive) are significantly superior to those of example 3 (without adhesive).

1. A pulverulent cosmetic and/or physiological composition comprising at least one binder and at least one pulverulent compound such that the binder comprises at least one adhesive material satisfying the following conditions:
G′ (2 Hz, 35° C.)≧10³ Pa, and
G′ (35° C.)≦10⁸ Pa, preferably G′ (35° C.)<10⁷ Pa,
G′ (2×10⁻² Hz, 35° C.)≦3×10⁵ Pa, and
G″/G′ (0.2 Hz, 35° C.)≧0.35
in which:
G′ (2 Hz, 35° C.) is the elastic shear modulus of said adhesive material, measured at a frequency of 2 Hz and at a temperature of 35° C.,
G′ (2×10⁻² Hz, 35° C.) is the elastic shear modulus of said adhesive material, measured at a frequency of 2×10⁻² Hz and at a temperature of 35° C.,
G′ (35° C.) is the elastic shear modulus of said adhesive material, measured at a temperature of 35° C. for any frequency between 2×10⁻² and 2 Hz,
G″(0.2 Hz, 35° C.) is the viscous shear modulus of said adhesive material, measured at a frequency of 0.2 Hz and at a temperature of 35° C.,
G′ (0.2 Hz, 35° C.) is the elastic shear modulus of said adhesive material, measured at a frequency of 0.2 Hz and at a temperature of 35° C.

2. The composition as claimed in the preceding claim such that:
G′ (2 Hz, 35° C.)≧10⁴ Pa.

3. The composition as claimed in either of the preceding claims, such that:
G′ (2×10⁻² Hz, 35° C.)≧5×10⁴ Pa.

4. The composition as claimed in any one of the preceding claims, such that:
G′ (2 Hz, 35° C.)≧10⁴ Pa, and
G′ (35° C.)≦10⁸ Pa, preferably G′(35° C.)≦10⁷ Pa,
G′ (2×10⁻² Hz, 35° C.)≦5×10⁴ Pa, and
G″/G′ (0.2 Hz, 35° C.)≧0.35.

5. The composition as claimed in any one of the preceding claims, such that the block and random copolymers comprising at least one monomer or a combination of monomers whose resultant polymer has a glass transition temperature of less than room temperature (25° C.), these monomers or combinations of monomers possibly being chosen from butadiene, ethylene, propylene, isoprene, isobutylene and a silicone, and blends thereof.

6. The composition as claimed in any one of the preceding claims, such that the adhesive materials are chosen from:
polyurethanes,
acrylic polymers,
silicones,
butyl rubbers, especially from polyisobutylenes,
ethylene/vinyl acetate polymers,
polyamides optionally modified with fatty chains,
natural rubbers,
and blends thereof.

7. The composition as claimed in any one of the preceding claims, such that the adhesive materials are chosen from polyisobutylenes with a relative molar mass Mv of greater than or equal to 10,000 and less than or equal to 150,000, and more preferably of greater than or equal to 18,000 and less than or equal to 150,000.

8. The composition as claimed in any one of the preceding claims, such that the adhesive material is a mixture of one or more polyisobutylenes with a relative molar mass of less than or equal to 10,000 and of one or more polyisobutylenes with a relative molar mass of greater than 150,000.

9. The composition as claimed in any one of the preceding claims, such that the adhesive materials are present in the composition according to the invention in a content ranging from 0.1% to 99%, preferably from 0.1% to 30% and more preferably from 0.1% to 10% by weight, relative to the total weight of the composition.

10. The composition as claimed in any one of the preceding claims, such that the binder comprises a fatty phase comprising at least one cosmetically or physiologically acceptable oil chosen especially from carbon-based, hydrocarbon-based and/or silicone-based oils of mineral, animal, plant or synthetic origin, and mixtures thereof.

11. The composition as claimed in the preceding claim, such that the fatty phase comprises at least one volatile oil.

12. The composition as claimed in the preceding claim, such that the weight ratio of volatile oil to the adhesive material ranges from 1 to 20, more preferably from 1 to 10 and better still from 2 to 4.

13. The composition as claimed in any one of the preceding claims, such that the fatty phase represents from 0.1% to 40%, of the total weight of the final composition and preferably from 0.1% to 20%.

14. The composition as claimed in any one of the preceding claims, such that the pulverulent compounds are chosen from pigments and/or nacres and/or fillers and/or mixtures thereof.

15. The composition as claimed in any one of the preceding claims, such that the pulverulent compounds represent from 60% to 99.9% and more preferably from 80% to 99.9% by weight, relative to the total weight of the composition.

16. The composition as claimed in any one of the preceding claims, such that the binder comprises an aqueous phase.

17. The use, in or for the manufacture of a cosmetic or physiological pulverulent composition, of at least one adhesive material that satisfies the following conditions:
G′ (2 Hz, 35° C.)≧10³ Pa, and
G′ (35° C.)≦10⁸ Pa, preferably G′ (35° C.)≦10⁷ Pa,
G′ (2×10⁻² Hz, 35° C.)≦3×10⁵ Pa, and
G″/G′ (0.2 Hz, 35° C.)≧0.35
in which:
G′ (2 Hz, 35° C.) is the elastic shear modulus of said adhesive material, measured at a frequency of 2 Hz and at a temperature of 35° C.,
G′ (2×10⁻² Hz, 35° C.) is the elastic shear modulus of said adhesive material, measured at a frequency of 2×10⁻² Hz and at a temperature of 35° C.,
G′ (35° C.) is the elastic shear modulus of said adhesive material, measured at a temperature of 35° C. for any frequency between 2×10⁻² and 2 Hz,
G′(0.2 Hz, 35° C.) is the viscous shear modulus of said adhesive material, measured at a frequency of 0.2 Hz and at a temperature of 35° C.,
G′ (0.2 Hz, 35° C.) is the elastic shear modulus of said adhesive material, measured at a frequency of 0.2 Hz and at a temperature of 35° C.,
to reduce, or even eliminate, the transfer and/or migration and/or to increase the staying power of the composition applied to human skin onto a support with which the deposit comes into contact.

18. The use, in or for the manufacture of a cosmetic or physiological pulverulent composition, of at least one adhesive material that satisfies the following conditions:
G′ (2 Hz, 35° C.)≧10³ Pa, and
G′ (35° C.)≦10⁸ Pa, preferably G′ (35° C.)≦10⁷ Pa,
G′ (2×10⁻² Hz, 35° C.)≦3×10⁵ Pa, and
G″/G′ (0.2 Hz, 35° C.)≧0.35
in which:
G′ (2 Hz, 35° C.) is the elastic shear modulus of said adhesive material, measured at a frequency of 2 Hz and at a temperature of 35° C.,
G′(2×10⁻² Hz, 35° C.) is the elastic shear modulus of said adhesive material, measured at a frequency of 2×10⁻² Hz and at a temperature of 35° C.,
G′ (35° C.) is the elastic shear modulus of said adhesive material, measured at a temperature of 35° C. for any frequency between 2×10⁻² and 2 Hz,
G″ (0.2 Hz, 35° C.) is the viscous shear modulus of said adhesive material, measured at a frequency of 0.2 Hz and at a temperature of 35° C.,
G′ (0.2 Hz, 35° C.) is the elastic shear modulus of said adhesive material, measured at a frequency of 0.2 Hz and at a temperature of 35° C., to increase the color fastness of the composition over time.

19. A nontherapeutic makeup and/or care process for human skin, which consists in applying to the skin a cosmetic composition as defined in any one of claims 1 to 16.
